(19) United States
(12) Patent Application Publication
(10) Pub. No.: US 2011/0158909 A1
(43) Pub. Date: Jun. 30, 2011

(54) Cationic Contrast Agents and Methods of Use Thereof

(75) Inventors: Mark W. Grinstaff, Brookline, MA (US); Neel Joshi, Cambridge, MA (US); Prashant Bansal, Quincy, MA (US)

(73) Assignee: Trustees of Boston University, Boston, MA (US)

(21) Appl. No.: 12/865,312

(22) PCT Filed: Jan. 27, 2009
(86) PCT No.: PCT/US2009/032125

§ 371 Date: Mar. 14, 2011

Related U.S. Application Data
(60) Provisional application No. 61/063,216, filed on Feb. 1, 2008.

(51) Int. Cl. A61K 49/00; C07C 237/46; A61P 29/00; A61P 23/00; A61P 31/00; A61P 31/12; A61P 31/10; A61P 31/04; A61P 39/06

(52) U.S. Cl. 424/9.1; 564/156; 564/153; 564/163

(57) ABSTRACT

The present invention provides compounds useful as contrast agents, such as for the CT imaging of cartilage tissue. The contrast agents are generally iodinated organic molecules that are positively charged under physiological environments. Also provided are compositions containing contrast agents and methods of using the agents, including, for example, the monitoring of glycosaminoglycan content in cartilage tissue. The invention provides non-invasive analytical techniques for the diagnosis of osteoarthritis in its earliest stages. The invention also provides improvements over existing contrast agents for cartilage monitoring, which tend to exhibit low residence times and require high dosages.

### RELATED APPLICATIONS

This application claims the benefit of priority to U.S. Provisional Patent Application Ser. No. 61/063,216, filed Feb. 1, 2008; the contents of which are hereby incorporated by reference.

### BACKGROUND OF THE INVENTION

Osteoarthritis (OA), a non-inflammatory joint disease characterized by degeneration of joint cartilage, can affect one or more parts of the body, including hands and weight-bearing joints such as knees, hips, feet and the spine. When healthy, cartilage allows bones to glide over each other and has a shock absorber function. The mechanical properties of cartilage are attributed to the unique and robust composition of its extracellular matrix (ECM). According to reports from the Arthritis Foundation, approximately 21 million people in the United States currently suffer from some form of OA. The exact causes of OA are still somewhat nebulous, but its onset is usually associated with age or excessive wear and tear of the joint. As the average age of the population in the United States continues to increase, the need for methods to counteract age-related diseases becomes more important.

Articular cartilage is made up of a proteoglycan extracellular matrix sparsely populated with chondrocytes. Its sturdiness and resistance to wear can be attributed to its layered structure (FIG. 1). In the deepest layer, collagen fibrils oriented perpendicular to the bone surface anchor the cartilage tissue firmly to the bone. The middle zone contains glycosaminoglycans (GAGs), which are responsible for the compressive stiffness of cartilage. At the surface of the cartilage tissue, collagen fibrils oriented parallel to the surface provide resistance to shear forces. In the early stages of OA, GAGs are proteolyzed and diffuse out of the extracellular matrix. This loss of GAGs in turn causes deterioration in the mechanical properties of the cartilage. Once the tissue is unable to withstand the high mechanical stresses of the joint, it degrades further, eventually leading to lesions and exposure of the bone surface and pain for the patient. Currently, osteoarthritis diagnosis relies mostly on observations of abnormal joint appearance (swelling), function (tenderness, loss of motion), and pain during physical examinations. These methods are only capable of diagnosing the disease in its later stages. In more extreme cases, the examining physician may turn to analysis of the synovial fluid or 2D radiography. However, the Arthritis Foundation reports that while most people over 60 show signs of OA according to x-rays, only ⅓ show actual symptoms. Synovial fluid analysis is a painful procedure and is inherently invasive.

Delayed Enhanced Magnetic Resonance Imaging of Cartilage (dGMERIC) is a technique for non-invasively monitoring the GAG content of articular cartilage. In this method, the patient is administered the contrast agent Gd(III)DOTA before imaging. Since the GAGs in cartilage ECM are highly negatively charged, the anionic contrast agent exhibits an inverse diffusion profile; that is, the concentration of contrast agent in the tissue is inversely proportional to the GAG concentration. Using this differential contrast agent diffusion, the relative GAG concentrations, and therefore the cartilage health, can be ascertained non-invasively. dGEMRIC use is still not very widespread. This is likely due to the limited availability of MR instruments and the high costs associated with their use. By contrast, computed tomography (CT) is cheaper, faster, and more easily accessible in hospitals across the country.

CT is a three-dimensional imaging modality that relies on the attenuation of x-ray radiation to generate images. It is one of the most commonly used techniques for medical imaging, and is employed for a range of diagnostic applications. The capability of CT to generate high resolution images of both soft and hard tissues makes it useful for investigating complex bone fractures, monitoring cancer progress in the abdomen, detecting pulmonary embolism, and intercranial brain hemorrhage, among other things. In order to obtain the best images, CT is often used in conjunction with a contrast agent. CT contrast agents usually include an inorganic or organic compound bearing heavy atoms capable of attenuating the x-ray intensity. The most common class of contrast agents stem from a triiodinated aromatic core that is further functionalized with carboxylic acid and amine derivatives.

CT used in conjunction with anionic iodinated contrast agents has already been shown to be an effective tool for imaging cartilage in a number of in vitro model systems. In these studies, the anionic contrast agent behaves analogously to the gadolinium contrast agent employed in the dGEMRIC technique. That is, x-ray attenuation coefficient for the cartilage tissue is inversely proportional to the GAG content due to the electrostatic repulsion between the contrast agent and the GAG molecules. Contrast agents that are capable of targeting specific organs or tissues are the lynchpin of modern medical imaging. The use of CT to diagnose the early stages of OA is just emerging as a clinically viable method. Consequently, there is a need for contrast agents capable of displaying meaningful interactions with cartilage tissue. Described herein is a class of cationic iodinated contrast agents.

Further objectives and advantages of the present invention will become apparent from the detailed description and claims.

### BRIEF DESCRIPTION OF THE FIGURES

FIG. 1a is a schematic illustration showing the synthesis of a diamine contrast agent (2). FIG. 1b depicts the structure of contrast agent 2 at pH 7 and below—under these conditions, the primary amines of the contrast agent are protonated, and therefore bear positive charges.

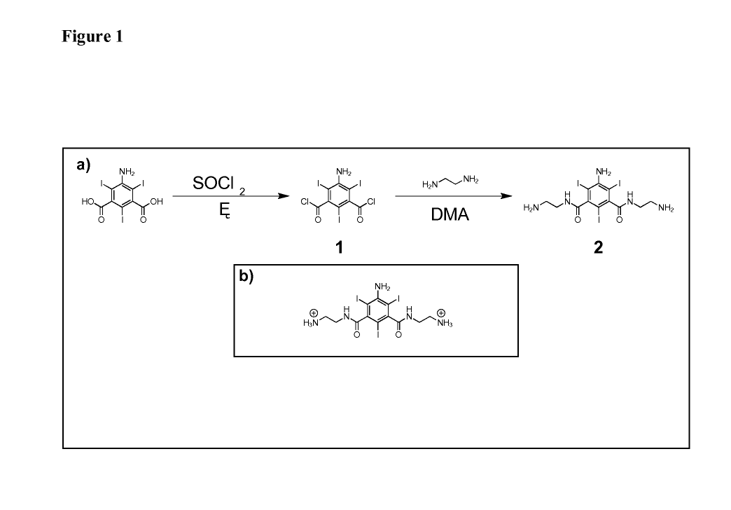

FIGS. 2a-d show experimental results of a contrast agent of the invention in osteochondral plugs. FIG. 2a is a photograph showing two opposing osteochondral plugs harvested from mature bovine knees. FIG. 2b is a CT image of an osteochondral plug. Areas of cartilage and bone are indicated. FIG. 2c is a graph demonstrating mean glycosaminoglycan (“GAG”) content of the plugs before (left, control) and after (right, degraded) degradation as determined by the DMMB assay. FIG. 2d is a graph showing a comparison of the changes in CT intensity after degradation for the anionic (left, positive) and the cationic (right, negative) contrast agents.

FIG. 3a is a schematic illustration showing the synthesis of a tetraamine-hexaiodo contrast agent (herein “contrast agent molecule 4”). FIG. 3b is a schematic illustration showing how, at pH 7 and below, the primary amines of contrast agent molecule 4 are protonated, and therefore bear positive charges.

FIG. 4a is a schematic illustration showing the synthesis of a monoamine-triiodo contrast agent (herein “contrast agent molecule 6”). FIG. 4b is a schematic illustration showing how, at pH 7 and below, the primary amine of contrast agent molecule 6 is protonated, and therefore bears a positive charge.

FIG. 5 depicts passive diffusion of contrast agents into and out of articular cartilage tissue. FIG. 5A depicts the average CT intensities of samples at various time points during immersion in contrast agent (iothalamate=triangles (approximately 500-600 average CT attenuation from time=5 h to time=24 h); 4=squares (approximately 700-900 average CT attenuation from time=5 h to time=24 h); 6=circles (approximately 900-1100 average CT attenuation from time=5 h to time=24 h); lines are meant only to guide the eye) and subsequent immersion in saline solution; n=4. FIG. 5B depicts representative CT images of samples during the time course study outlined in FIG. 5A. In all images, the dark lower layer indicates bone (high CT attenuation). The lighter, upper layer indicates cartilage at various levels of CT attenuation. For example, at 0 h (left hand column), the cartilage layers reflect a low level of CT attenuation. At 2 h, both contrast agent 6 and 4 increased the CT attenuation of the cartilage to medium levels. Iothalamate showed very little change in CT attenuation at t=2 h. Additionally, after 2 h of immersion in saline, contrast agent 6 showed similar attenuations as after 24 h of immersion in contrast agent. At this time, the attenuations of both contrast agent 4 and iothalamate began to decline. After 24 h in saline, both contrast agent 4 and iothalamate reflected CT attenuations similar to t=0, while contrast agent 6 still showed medium levels of CT attenuation, especially close to the bone surface.

FIG. 6 depicts a linear regression analysis of initial diffusion rates. The first three data points (0, 1, and 2 h) from each of the data sets in FIG. 5A were averaged across samples and subjected to regression analysis (iothalamate=triangles, bottom line; 4=squares, top line; 6=circles; middle line). The values for slope, y-intercept, and R² for each contrast agent group are reported in Table 1. Cationic contrast agents 4 and 6 diffuse into cartilage 1.77 and 1.65 times faster, respectively, than the anionic iothalamate.

FIG. 7 depicts data from a cartilage degradation study. FIG. 7A depicts the linear regression analysis of average CT intensity vs. GAG content for three different contrast agents (iothalamate−negative slope, bottom line; 6−slope=13758, top line; 4−slope=4688, middle line). GAG content reported as 100×(mg of GAG)/(mg of hydrated cartilage). Dotted lines represent 95% confidence intervals of the mean. FIG. 7B depicts representative CT images of degraded and undegraded osteochondral plugs after equilibrium in three different contrast agents. The low levels of CT attenuation observed with iothalamate do not change significantly between the degraded and the undegraded samples. Contrast agent 4 produces higher levels of CT attenuation than iothalamate. However, contrast agent 6 produces high levels of CT attenuation. This is especially evident in the undegraded sample. Additionally, increased diffusion of the cationic contrast agents into the intact cartilage with high GAG concentration and less diffusion in the GAG depleted cartilage is observed. The CT attenuation pattern also reflects the diffusion of the cationic contrast agents in proportion to the GAG content of the degraded cartilage. The decreased CT attenuation in the superficial zone cartilage reflects the extent that the chondroitinase was able to penetrate the cartilage and degrade the proteoglycans. All three images in each column come from the same sample.

FIG. 8 depicts microCT imaging of rabbit femur with cationic contrast agent 6. FIG. 8A depicts an axial slice of femoral head. The white box indicates area depicted in zoomed-in view B. FIG. 8C depicts sagittal slice of medial condyle. White box indicates area depicted in zoomed-in view D. FIG. 8E depicts a histological analysis of rabbit femur stained with GAG-specific dye Safranin-O (red indicates higher GAG content). FIG. 8F depicts the thickness map of articular cartilage.

FIG. 9a is a graph showing the effect of trypsin degradation on CT intensity. FIG. 9b is a graph showing the effect of trypsin degradation on total residual GAG content. FIG. 9c is a reproduction of a CT image showing segmented articular cartilage. Segmented cartilage surface is shown, and lines the surface of the humerus. Segmented cartilage surface is shown in red.

FIG. 10 depicts the synthesis of the multi-cationic, dendritic contrast agents possessing six iodine atoms per molecule (9 and 10).

FIG. 11 depicts exemplary multi-cationic, dendritic contrast agents possessing three iodine atoms per molecule (11, 12, 13, and 14).

FIG. 12 depicts the synthesis of hydroxylated cationic contrast agent 18, which possesses four positive charges upon protonation (19).

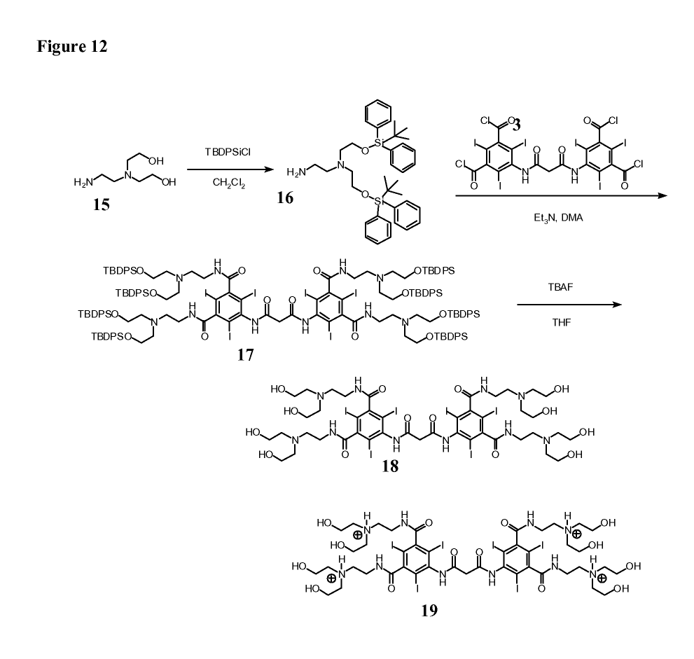

### DETAILED DESCRIPTION

Unless defined otherwise, all technical and scientific terms used herein have the same meaning as commonly understood by one of ordinary skill in the art to which this invention belongs. Where a term is provided in the singular, the inventor also contemplates the plural of that term. The nomenclature used herein and the procedures described below are those well known and commonly employed in the art.

The present invention provides a new class of contrast agents, suitable for imaging applications such as x-ray imaging. These contrast agents are organic molecules of varying chemical compositions and sizes. The molecules act as contrast agents by virtue of incorporating atoms that are capable of modulating x-ray attenuation coefficients. The contrast agents are charged (either anionic or cationic) at physiologically relevant pHs (typically in the range of 6.0-8.0). The charged contrast agents exhibit a cartilage tissue diffusion profile that is dependent on the GAG content of the extracellular matrix. Since the diffusion of the contrast agents is sensitive to GAG content, it will be a critical component to a new class of medical procedures that use CT imaging to diagnose OA at its earliest stages.

The contrast agents provided for in this invention include organic molecules that bear one or more positive charges. Exemplary contrast agents to be described in detail include contrast agents with one of the following structures:

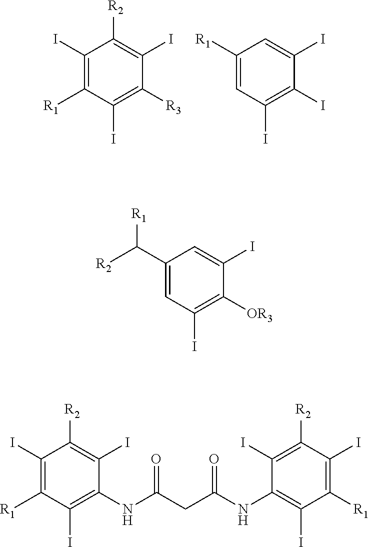

R₁, R₂, and R₃ are either the same or different and may contain H, alkyl, alkenyl, alkynyl, OH, OR′, COOR′, OCOOR′, CONHR′, OCOONHR′, CONHR′₂, NHCONHR′, NHCSNHR′, OCSNHR′, NH₂, NHR′, or NR′₂, or any combination thereof. Each occurrence of R′ is independently H, an alkyl, an alkenyl, an alkynyl, (CH₂)ₙOH, (CHR″)ₙCH₂R″, (CH₂)ₙNH₂, (CH₂)ₙOR″, (CH₂)ₙNHR″, (CH₂)ₙCOOH, (CH₂)ₙCOOR, CO(CH₂)ₙOR″, COO(CH₂)ₙOR″, COCH(OR″)(CH₂)ₙOR″, COOCH(OR″)(CH₂)ₙOR″, (CHR″)ₙR″, an amino acid, a peptide, a carbohydrate a synthetic polymer such as poly(ethylene glycol) or polyacrylate and n is an integer, generally from 1 to 2000. Each occurrence of R″ is independently H, an alkyl, an alkenyl, an alkynyl, (CH₂)ₙOH, (CH₂)ₙNH₂, OH, OR′″, NH₂, NR′″, SH, SR′″, COOH, COOR″′, CONH₂, (CH₂)ₙNR″′₂, (CH₂)ₙCOOH, (CH₂)ₙCOOR′″, CO(CH₂)ₙOR″, COCH(OR″)(CH₂)ₙOR″, OOCCH₃, an amino acid, a small or large peptide, a carbohydrate a synthetic polymer such as poly(ethylene glycol) or polyacrylate. Generally, n is an integer from 1 to 2000.

The invention provides linear or branched oligomers of the contrast agents. These structures may be symmetric or asymmetric. For example, as shown below:

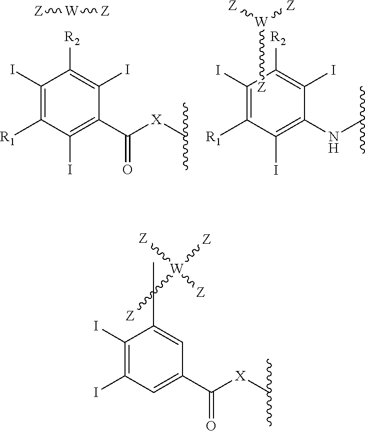

each occurrence of Z is independently selected form the following iodinated ring structures:
R₁ and R₂ are either the same or different and may include H, COOR′, CONHR′, CONHR′₂, NH₂, NHR′, or NHR′₂. Each occurrence of R′ is independently H, an alkyl, an alkenyl, an alkynyl, (CH₂)ₙOH, (CHR″)ₙCH₂R″, (CH₂)ₙNH₂, (CH₂)ₙOR″, (CH₂)ₙNHR″, (CH₂)ₙCOOH, (CH₂)ₙCOOR, CO(CH₂)ₙOR″, COO(CH₂)ₙOR″, COCH(OR″)(CH₂)ₙOR″, COOCH(OR″)(CH₂)ₙOR″, (CHR″)ₙR″, an amino acid, a peptide, or a carbohydrate, and n is an integer from 1 to 2000. Each occurrence of R″ is independently H, an alkyl, an alkenyl, an alkynyl, (CH₂)ₙOH, (CH₂)ₙNH₂, OH, OR″′, NH₂, NR′″, SH, SR′″, COOH, COOR″′, CONH₂, (CH₂)ₙNR″′₂, (CH₂)ₙCOOH, (CH₂)ₙCOOR″′, CO(CH₂)ₙOR″, COCH(OR″)(CH₂)ₙOR″, OOCCH₃, amino acid, a peptide, or a carbohydrate, and n is an integer from 1 to 2000. X is O, S, or NH, and W is a linking moiety.

The invention provides linking moieties for the cationic contrast agents. Preferably, the linking moiety contains a covalent linkage bond. For example, W may be a chemical functionality possessing an amide bond, carbamate bond, urea bond, thiourea, Schiff base bond, peptide ligation, and carbon-carbon bond. For example, W may include of one of the structures shown below:

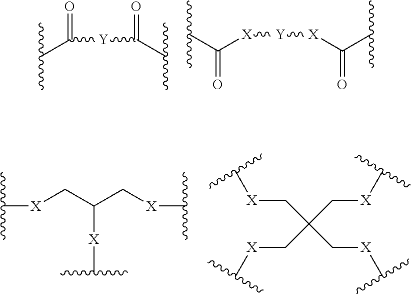

wherein each occurrence of Y may be an alkyl, poly(ethylene glycol), polyethylene, X(CH₂CH₂X)ₙ, or X((CH₂)ₘX)ₙ, and each occurrence of X may be independently S, O, NH, SH, OH, or NH₂, and m and n are integers ranging from 1 to 2000.

The linking moiety for the cationic contrast agent may include COO(CH₂CH₂O)ₙCOO, CONR′(CH₂CH₂O)ₙCONR′, CO(CH₂CH₂O)ₙCO, COO(CH₂CH₂O)ₙCO, CONR′(CH₂CH₂O)ₙCO, (OCH₂CH₂)ₙO, (OCH₂CH₂)ₙNR′, NR′CH₂CH₂(OCH₂CH₂)ₙNR′, CH₂CH₂(OCH₂CH₂)ₙ, or CH₂CH₂(OCH₂CH₂)ₙ, where each occurrence of R′ is independently H, an alkyl, an alkenyl, an alkynyl, (CH₂)ₙOH, (CH₂)ₙNH₂, COOH, CONH₂, an amino acid, a peptide, or a carbohydrate, and n is an integer from 1 to 2000.

The structures described herein may be covalently linked to form oligomers or polymers of iodinated structures. These structures are linear or dendritic and can be connected by a linker moiety and may be either symmetric or asymmetric. Oligomeric or polymeric versions of the iodinated molecules will allow for the same concentrations of organically bound iodine at lower molar concentrations. The size of the molecule modulates its diffusion through cartilage tissue, thereby affecting the quality of the CT images.

The contrast agent may be combined with one or more materials in various relative amounts in order to make a formulation. For example, a formulation is provided wherein the organic molecule is combined with a viscous polymer such as hyaluronic acid or a synthetic polymer. These polymers generally have a viscosity greater than about 0.1 Pa·s and less than about 10000 Pa·s. The present invention also provides for anionic contrast agents that are combined with one or more viscosity-enhancing substances. The perpetual circulation and replenishment of synovial fluid may limit the residence time of the contrast agent in the joint space. The administration of a formulation that contains a combination of a contrast agent and a viscosity-enhancing agent will prolong the lifetime of the contrast agent in the area of the joint space, allowing the contrast agent more time to effectively diffuse into the cartilage tissue, resulting in higher intensity CT images. The viscosity enhancing agent described herein may include of a biopolymer such as hyaluronic acid of molecular weight >500 KDa. The high molecular weight hyaluronic acid may also be covalently crosslinked to produce a more robust substance. The hyaluronic acid may also be crosslinked through non-covalent bonds, such as ionic bonds, hydrogen bonds, or ligand-receptor binding. The viscosity-enhancing agent may also contain of a synthetic polymer. The polymer may be, e.g., poly(ethylene glycol), polyacrylate, polysaccharides, poly(amino acid), or ring-opening metathesis polymer.

In other embodiments, the formulation additionally contains a vasoconstrictor, such as epinephrine. In general, a vasoconstrictor temporarily inhibits the passive diffusion or active removal of the contrast agent away from the joint space, allowing a period of time that generally exceeds a period of time in the absence of the vasoconstrictor for the contrast agent to diffuse into the cartilage tissue. The administration of the vasoconstrictor may be concurrent with that of the contrast agent or at another time during the treatment (e.g., either before or after administration of the contrast agent).

The contrast agents may be covalently attached to a biomolecule such as an antibody, hormone, peptide, protein, DNA, RNA, carbohydrate, or a polysaccharide. Alternatively, the contrast agents may be covalently attached to or non-covalently encapsulated by a synthetic polymer or other macromolecule. This embodiment may take the form of a dendritic or linear structure. Optionally, the contrast agents described herein are covalently attached to a biomolecule such as an antibody, hormone, peptide, protein, DNA, RNA, carbohydrate, or polysaccharide.

### DEFINITIONS

Throughout the specification, several terms are employed that are defined in the following paragraphs.

The terms “individual” and “subject” are used herein interchangeably. They refer to a human or another mammal (e.g., primates, dogs, cats, goats, horses, pigs, mice, rabbits, and the like). In certain preferred embodiments, the subject is human. The terms do not denote a particular age, and thus encompass adults, children, and newborn.

The term “treatment” is used herein to characterize a method or process that is aimed at (1) delaying or preventing the onset of a disease or condition; (2) slowing down or stopping the progression, aggravation, or deterioration of the symptoms of the disease or condition; (3) bringing about ameliorations of the symptoms of the disease or condition; or (4) curing the disease or condition. A treatment may be administered prior to the onset of the disease, for a prophylactic or preventive action. Alternatively or additionally, the treatment may be administered after initiation of the disease or condition, for a therapeutic action.

The term “local”, when used herein to characterize the delivery, administration or application of a polymer of the present invention, or a pharmaceutical composition thereof, is meant to specify that the polymer or composition, is delivered, administered or applied directly to the site to be treated or in the vicinity of the site to be treated for a localized effect. For example, a polysaccharide mimic used as a viscosupplement will generally be injected directly to an osteoarthritic knee joint; a polysaccharide mimic used as tissue space filler will generally be injected directly to a diseased or damaged vocal cord, or to a skin area displaying lines or wrinkles. Preferably, local administration is effected without any significant absorption of components of the polysaccharide mimic into the patient's blood stream (to avoid a systemic effect).

A “pharmaceutical composition” is defined herein as comprising an effective amount of at least one active ingredient (e.g., a polysaccharide mimic), and at least one pharmaceutically acceptable carrier.

As used herein, the term “pharmaceutically acceptable carrier” refers to a carrier medium which does not interfere with the effectiveness of the biological activity of the active ingredient(s) and which is not excessively toxic to the host at the concentration at which it is administered. The term includes solvents, dispersion media, coatings, antibacterial and antifungal agents, isotonic agents, absorption delaying agents, and the like. The use of such media and agents for pharmaceutically active substances is well known in the art (see for example, “Remington's Pharmaceutical Sciences”, E. W. Martin, 18^{th} Ed., 1990, Mack Publishing Co.: Easton, Pa., which is incorporated herein by reference in its entirety).

As used herein, the term “effective amount” refers to any amount of a molecule/macromolecule, compound or composition that is sufficient to fulfill its intended purpose(s), i.e., to elicit a desired biological or medicinal response in a tissue or subject. Examples of intended purposes of a contrast agent include, but are not limited to, to monitor GAG content in articular cartilage, or to enhance x-ray based images of connective tissue obtained from CT or plane radiographs.

As used herein, the term “connective tissue” includes all musculoskeletal tissue of the body. Examples of connective tissue include, but are not limited to, muscles, tendons, fibrous tissues, fat, and synovial tissues.

The terms “bioactive agent” and “biologically active agent” are used herein interchangeably. They refer to compounds or entities that alter, inhibit, activate or otherwise affect biological or chemical events. For example, bioactive agents may include, but are not limited to, vitamins, anti-cancer substances, antibiotics, immunosuppressants, anti-viral substances, enzyme inhibitors, opioids, hypnotics, lubricants, tranquilizers, anti-convulsants, muscle relaxants, anti-spasmodics and muscle contractants, anti-glaucoma compounds, modulators of cell-extracellular matrix interactions including cell growth inhibitors and anti-adhesion molecules, vasodilating agents, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, imaging agents. A more complete, although not exhaustive, listing of classes and specific drugs suitable for use in the present invention may be found in “Pharmaceutical Substances: Synthesis, Patents, Applications” by A. Kleeman and J. Engel, Thieme Medical Publishing, 1999; and the “Merck Index: An Encyclopedia of Chemicals, Drugs, and Biologicals”, S. Budavari et al. (Eds), CRC Press, 1996, both of which are incorporated herein by reference.

The term “small molecule” refers to molecules, whether naturally-occurring or artificially created (e.g., via chemical synthesis) that have a relatively low molecular weight. Preferred small molecules are biologically active in that they produce a local or systemic effect in animals, preferably mammals, more preferably humans. Typically, small molecules have a molecular weight of less than about 1,500 Da. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered suitable for use with the present cationic contrast agents.

An entity is herein said to be “associated with” another entity if they are linked by a direct or indirect, covalent or non-covalent interaction. In certain embodiments, the association is covalent. Desirable non-covalent interactions include hydrogen bonding, van der Walls interactions, hydrophobic interactions, magnetic interactions, electrostatic interactions, or combinations thereof.

In general, the term “aliphatic”, as used herein, includes both saturated and unsaturated, straight chain (i.e., unbranched) or branched aliphatic hydrocarbons, which are optionally substituted with one or more functional groups, as defined below. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having 1-6 carbon atoms. In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms.

Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents, as previously defined. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alicyclic”, as used herein, refers to compounds which combine the properties of aliphatic and cyclic compounds and include but are not limited to cyclic, or polycyclic aliphatic hydrocarbons and bridged cycloalkyl compounds, which are optionally substituted with one or more functional groups, as defined below. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl-n, cyclohexyl, —CH₂-cyclohexyl, cyclohexenylethyl, cyclohexanylethyl, norborbyl moieties and the like, which again, may bear one or more substituents.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen sulfur, nitrogen, phosphorus or silicon atoms, e.g., in place of carbon atoms. Heteroaliphatic moieties may be saturated or unsaturated, branched or linear (i.e., unbranched), and substituted or unsubstituted. Substituents include, but are not limited to, any of the substituents mentioned below, i.e., the substituents recited below resulting in the formation of a stable compound.

The term “heteroalicyclic”, as used herein, refers to compounds which combine the properties of heteroaliphatic and the cyclic compounds and include but are not limited to saturated and unsaturated mono- or polycyclic heterocycles such as morpholino, pyrrolidinyl, furanyl, thiofuranyl, pyrrolyl, etc, which are optionally substituted with one or more functional groups. Substituents include, but are not limited to, any of the substituents mentioned below, i.e., the substituents recited below resulting in the formation of a stable compound.

The term “alkyl”, as used herein, refers to saturated, straight- or branched-chain hydrocarbon radicals derived from a hydrocarbon moiety containing between one and twenty carbon atoms by removal of a single hydrogen atom, which alkyl groups are optionally substituted with one or more functional groups. Substituents include, but are not limited to, any of the substituents mentioned below, i.e., the substituents recited below resulting in the formation of a stable compound. Examples of alkyl radicals include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The term “alkoxy”, as used herein, refers to an alkyl group, as previously defined, attached to the parent molecular moiety through an oxygen atom. Examples include, but are not limited to, methoxy, ethoxy, propoxy, isopropoxy, n-butoxy, tert-butoxy, neopentoxy, and n-hexoxy.

The term “alkenyl” denotes a monovalent group derived from a hydrocarbon moiety having at least one carbon-carbon double bond, which alkenyl group is optionally is substituted with one or more functional groups. In certain embodiments, an alkenyl group contains between one and twenty carbon atoms. Substituents include, but are not limited to, any of the substituents mentioned below, i.e., the substituents recited below resulting in the formation of a stable compound. Alkenyl groups include, for example, ethenyl, propenyl, butenyl, 1-methyl-2-buten-1-yl, and the like.

The term “alkynyl”, as used herein, refers to a monovalent group derived from a hydrocarbon having at least one carbon-carbon triple bond, which alkynyl group is optionally substituted. In certain embodiments, an alkynyl group contains between one and twenty carbon atoms. Substituents include, but are not limited to, any of the substituents mentioned below, i.e., the substituents recited below resulting in the formation of a stable compound. Representative alkynyl groups include ethynyl, 2-propynyl (propargyl), 1-propynyl, and the like.

The term “amine”, as used herein, refers to one, two, or three alkyl groups, as previously defined, attached to the parent molecular moiety through a nitrogen atom. The term “alkylamino” refers to a group having the structure —NHR′ wherein R′ is an alkyl group, as previously defined; and the term “dialkylamino” refers to a group having the structure —NR′R″, wherein R′ and R″ are each independently alkyl groups. The term “trialkylamino” refers to a group having the structure —NR′R″R′″, wherein R′, R″, and R′″ are each independently alkyl groups. Additionally, R′, R″, and/or R′″ taken together may optionally be —(CH₂)ₖ— where k is an integer from 2 to 6. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “heteroaryl”, as used herein refers to a stable heterocyclic or polyheterocyclic, unsaturated radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms. Heteroaryl moieties may be substituted or unsubstituted. Substituents include, but are not limited to, any of the substituents mentioned below, i.e., the substituents recited below resulting in the formation of a stable compound. Examples of heteroaryl nuclei include pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

The term “aryl”, as used herein, refers to stable mono- or polycyclic, unsaturated moieties having preferably 3-14 carbon atoms, each of which may be substituted or unsubstituted. Substituents include, but are not limited to, any of the substituents mentioned below, i.e., the substituents recited below resulting in the formation of a stable compound. The term aryl may refer to a mono- or bicyclic carbocyclic ring system having one or two aromatic rings including, but not limited to, phenyl, naphthyl, tetrahydronaphthyl, indanyl, indenyl and the like.

It will also be appreciated that aryl and heteroaryl moieties, as defined herein, may be attached via an aliphatic, alicyclic, heteroaliphatic, heteroalicyclic, alkyl or heteroalkyl moiety and thus also include -(aliphatic)aryl, -(heteroaliphatic)aryl, -(aliphatic)heteroaryl, -(heteroa-liphatic)heteroaryl, -(alkyl)aryl, -(heteroalkyl)aryl, -(heteroalkyl)aryl, and -(heteroalkyl)-heteroaryl moieties. Thus, as used herein, the phrases “aryl or heteroaryl” and “aryl, heteroaryl, -(aliphatic)aryl, -(heteroaliphatic)aryl, -(aliphatic)heteroaryl, -(heteroaliphatic)heteroaryl, -(alkyl)aryl, -(heteroalkyl)aryl, -(heteroalkyl)aryl, and -(heteroalkyl)heteroaryl” are interchangeable.

The term “carboxylic acid”, as used herein, refers to a group of formula —CO₂H.

The terms “halo”, “halide”, and “halogen”, as used herein, refers to an atom selected from fluorine, chlorine, bromine, and iodine.

The term “mercaptoalkyl”, a used herein, refers to an alkyl group, as defined above, bearing at least one SH group.

The term “heterocyclic”, as used herein, refers to a non-aromatic partially unsaturated or fully saturated 3- to 10-membered ring system, which includes single rings of 3 to 8 atoms in size and bi- and tri-cyclic ring systems which may include aromatic six-membered aryl or aromatic heterocyclic groups fused to a non-aromatic ring. Heterocyclic moieties may be substituted or unsubstituted. Substituents include, but are not limited to, any of the substituents mentioned below, i.e., the substituents recited below resulting in the formation of a stable compound. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized.

The term “acyl”, as used herein, refers to a group comprising a carbonyl group of the formula C═O. Examples of acyl groups include aldehydes, ketones, carboxylic acids, acyl halides, anhydrides, thioesters, amides, urea, carbamate, and carboxylic esters.

The term “hydrocarbon”, as used herein, refers to any chemical group comprising hydrogen and carbon. The hydrocarbon may be substituted or unsubstituted. The hydrocarbon may be unsaturated, saturated, branched, unbranched, cyclic, polycyclic, or heterocyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, and the like. As would be known to one skilled in this art, all valencies must be satisfied in making any substitutions. Likewise a fluorocarbon as used herein refers to any chemical group comprising more fluorine than hydrogen with carbon. hydrocarbon may be substituted or unsubstituted. The fluorocarbon may be unsaturated, saturated, branched, unbranched, cyclic, polycyclic, or heterocyclic.

The term “substituted”, whether preceded by the term “optionally” or not, refers to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Heteroatoms such as nitrogen may have hydrogen substituents and/or any permissible substituents of organic compounds described herein which satisfy the valencies of the heteroatoms. Examples of substituents include, but are not limited to aliphatic; alicyclic; heteroaliphatic; heteroalicyclic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —NCO—CF₃; —CH₂CF₃; —CHCl₂; —CH₂ORₓ; —CH₂CH₂ORₓ; —CH₂N(Rₓ)₂; —CH₂SO₂CH₃; —C(O)Rₓ; —CO₂(Rₓ); —CON(Rₓ)₂; —OC(O)Rₓ; —C(O)OC(O)Rₓ; —OCO₂Rₓ; —OCON(Rₓ)₂; —N(Rₓ)₂; —S(O)₂Rₓ; —NRₓ(CO)Rₓ, —NRₓ(CO)N(Rₓ)₂ wherein each occurrence of Rₓ independently includes, but is not limited to, H, aliphatic, alicyclic, heteroaliphatic, heteroalicyclic, aryl, heteroaryl, alkylaryl, or alkylheteroaryl, wherein any of the aliphatic, alicyclic, heteroaliphatic, heteroalicyclic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted.

The term “dendrimer” refers to repeatedly-branched species that are characterized by their structure perfection. Structural perfection is based on the evaluation of both symmetry and polydispersity. The term “dendrimer” is meant to encompass both low-molecular weight and high-molecular weight species. Low molecular weight dendrimers include, but are not limited to, dendrimers and dendrons. High-molecular weight dendrimers include, but are not limited to, dendritic polymers, dendronized polymers, hyperbranched polymers, and brush-polymers.

### Exemplary Uses and Applications of the Cationic Contrast Agents

The contrast agents are used in the imaging of cartilage in articular joints. These joints include knees, hips, elbows, wrists, etc. The administration of the contrast agent will be performed via intra-articular injection. Alternative routes of administration include intravenous, intraperitoneal, dermal, intradermal, intramuscular and/or subcutaneous injections. The contrast agents, once in the joint space, diffuse into the cartilage tissue due to the electrostatic attraction between the anionic GAGs of the cartilage ECM and the positive charge of the contrast agent. The extent to which the contrast agents diffuse into the cartilage tissue is directly proportional to the GAG content, and is therefore a useful indicator of cartilage health.

Since the success of the imaging is dependent upon the ability for the contrast agent to passively diffuse into the cartilage tissue, any means to maintain a high concentration of contrast agent in the joint space will naturally result in better quality CT images. The perpetual circulation and replenishment of synovial fluid limits the residence time of the contrast agent in the joint space. Therefore, the administration of a formulation including of the combination of a contrast agent and a viscosity-enhancing agent will prolong the lifetime of the contrast agent in the joint space, allowing it more time to effectively diffuse into the cartilage tissue, resulting in higher intensity CT images.

In one embodiment, the invention relates to a method of assessing the concentration of GAG in mammalian cartilage, comprising the steps of acquiring a first x-ray radiographic image of a mammalian joint; injecting a contrast agent into said joint; acquiring a second x-ray radiographic image of said joint; and comparing said first image to said second image.

In one embodiment, the invention relates to a method of assessing the mechanical properties of mammalian cartilage, comprising the steps of acquiring a first x-ray radiographic image of a mammalian joint; injecting a contrast agent into said joint; acquiring a second x-ray radiographic image of said joint; and comparing said first image to said second image.

In one embodiment, the invention relates to a method of treatment of an individual based on the CT data and analysis of an imaged joint containing one or more contrast agents.

### Dosages and Administration

In a method of treatment of the present invention, a molecule/macromolecule, or a pharmaceutical composition thereof, will generally be administered in such amounts and for such a time as is necessary or sufficient to achieve at least one desired result.

A treatment according to the present invention may include of a single dose or a plurality of doses over a period of time. Administration may be one or multiple times daily, weekly (or at some other multiple day interval) or on an intermittent schedule.

Contrast agents of the present invention, or compositions thereof, may be administered using any route of administration effective for achieving the desired effect. The compositions when used for administration into a subject in need thereof are of a pharmaceutically acceptable nature. Administration will generally be local rather than systemic. Methods of local administration include, but are not limited to, dermal, intradermal, intramuscular, intraperitoneal, subcutaneous, and intra-articular routes.

Depending on the route of administration, effective doses may be calculated according to the body weight, body surface area, or organ size of the subject to be treated. Optimization of the appropriate dosages can readily be made by one skilled in the art in light of pharmacokinetic data observed in human clinical trials. Alternatively or additionally, the dosage to be administered can be determined from studies using animal models for the particular type of condition to be treated, and/or from animal or human data obtained from agents which are known to exhibit similar pharmacological activities. The final dosage regimen will be determined by the attending surgeon or physician, considering various factors which modify the action of active agent, e.g., the agent's specific activity, the agent's specific half-life in vivo, the severity of the condition and the responsiveness of the patient, the age, condition, body weight, sex and diet of the patient, the severity of any present infection, time of administration, the use (or not) of other concomitant therapies, and other clinical factors.

### Pharmaceutical Compositions

As mentioned above, methods of treatment of the present invention include administration of a contrast agent per se or in the form of a pharmaceutical composition. A pharmaceutical composition will generally comprise an effective amount of at least one polymer and at least one pharmaceutically acceptable carrier or excipient.

Pharmaceutical compositions of the present invention may be formulated according to general pharmaceutical practice (see, for example, “Remington's Pharmaceutical Sciences” and “Encyclopedia of Pharmaceutical Technology”, J. Swarbrick, and J. C. Boylan (Eds.), Marcel Dekker, Inc: New York, 1988). The optimal pharmaceutical formulation can be varied depending upon the route of administration and desired dosage. Such formulations may influence the physical state, stability, rate of in vivo release, and rate of in vivo clearance of the administered compounds. Formulation will preferably produce liquid or semi-liquid (e.g., gel) pharmaceutical compositions.

Pharmaceutical compositions may be formulated in dosage unit form for ease of administration and uniformity of dosage. The expression “unit dosage form”, as used herein, refers to a physically discrete unit of cationic contrast agent for the patient to be treated. Each unit contains a predetermined quantity of active material calculated to produce the desired effect. It will be understood, however, that the total dosage of the composition will be decided by the attending physician within the scope of sound medical judgment.

### Formulation

Physiologically acceptable carriers, vehicles, and/or excipients for use with pharmaceutical compositions of the present invention can be routinely selected for a particular use by those skilled in the art. These include, but are not limited to, solvents, buffering agents, inert diluents or fillers, suspending agents, dispersing or wetting agents, preservatives, stabilizers, chelating agents, emulsifying agents, anti-foaming agents, ointment bases, penetration enhancers, humectants, emollients, and skin protecting agents.

Examples of solvents include water, Ringer's solution, U.S.P., isotonic sodium chloride solution, alcohols, vegetable, marine and mineral oils, polyethylene glycols, propylene glycols, glycerol, and liquid polyalkylsiloxanes. Inert diluents or fillers may be sucrose, sorbitol, sugar, mannitol, microcrystalline cellulose, starches, calcium carbonate, sodium chloride, lactose, calcium phosphate, calcium sulfate, or sodium phosphate. Examples of buffering agents include citric acid, acetic acid, lactic acid, hydrogenophosphoric acid, and diethylamine. Suitable suspending agents include, for example, naturally-occurring gums (e.g., acacia, arabic, xanthan, and tragacanth gum), celluloses (e.g., carboxymethyl-, hydroxyethyl-, hydroxypropyl-, and hydroxypropylmethylcellulose), hyaluronic acid, alginates and chitosans. Examples of dispersing or wetting agents are naturally-occurring phosphatides (e.g., lecithin or soybean lecithin), condensation products of ethylene oxide with fatty acids or with long chain aliphatic alcohols (e.g., polyoxyethylene stearate, polyoxyethylene sorbitol monooleate, and polyoxyethylene sorbitan monooleate). Also included are mixtures of one or more of these solvents.

Preservatives may be added to a pharmaceutical composition of the present invention to prevent microbial contamination that can affect the stability of the formulation and cause infection in the patient. Suitable examples of preservatives include parabens (such as methyl-, ethyl-, propyl-, p-hydroxy-benzoate, butyl-, isobutyl- and isopropyl-paraben), potassium sorbate, sorbic acid, benzoic acid, methyl benzoate, phenoxyethanol, bronopol, bronidox, MDM hydantoin, iodopropylnyl butylcarbamate, benzalconium chloride, cetrimide, and benzylalcohol. Examples of chelating agents include sodium EDTA and citric acid. Also included are mixtures of one or more of these preservatives.

Examples of emulsifying agents are naturally-occurring gums, naturally-occurring phosphatides (e.g., soybean lecithin, sorbitan mono-oleate derivatives), sorbitan esters, monoglycerides, fatty alcohols, and fatty acid esters (e.g., triglycerides of fatty acids). Anti-foaming agents usually facilitate manufacture, they dissipate foam by destabilizing the air-liquid interface and allow liquid to drain away from air pockets. Examples of anti-foaming agents include simethicone, dimethicone, ethanol, and ether. Also included are mixtures of one or more of these emulsifying agents.

Examples of gel bases or viscosity-increasing agents are liquid paraffin, polyethylene, fatty oils, colloidal silica or aluminum, glycerol, propylene glycol, carboxyvinyl polymers, magnesium-aluminum silicates, hydrophilic polymers (such as, for example, starch or cellulose derivatives), water-swellable hydrocolloids, carragenans, hyaluronates, and alginates. Ointment bases suitable for use in the pharmaceutical compositions of the present invention may be hydrophobic or hydrophilic; and specific examples include paraffin, lanolin, liquid polyalkylsiloxanes, cetanol, cetyl palmitate, vegetable oils, sorbitan esters of fatty acids, polyethylene glycols, and condensation products between sorbitan esters of fatty acids, ethylene oxide (e.g., polyoxyethylene sorbitan monooleate), and polysorbates. Also included are mixtures of one or more of these gel bases or viscosity-increasing agents.

Examples of humectants are ethanol, isopropanol glycerin, propylene glycol, sorbitol, lactic acid, and urea. Suitable emollients include cholesterol and glycerol. Examples of skin protectants include vitamin E, allatoin, glycerin, zinc oxide, vitamins, and sunscreen agents. Also included are mixtures of one or more of these humectants.

In certain embodiments, pharmaceutical compositions of the present invention may, alternatively or additionally, comprise other types of excipients including, thickening agents, bioadhesive polymers, and permeation enhancing agents. Also included are mixtures of one or more of these excipients.

Thickening agents are generally used to increase viscosity and improve bioadhesive properties of pharmaceutical compositions. Examples of thickening agents include, but are not limited to, celluloses, polyethylene glycol, polyethylene oxide, naturally occurring gums, gelatin, karaya, pectin, alginic acid, and povidone. In certain embodiments, a thickening agent is selected for its thioxotropic properties (i.e., has a viscosity that is decreased by shaking or stirring). The presence of such as an agent in a pharmaceutical composition allows the viscosity of the composition to be reduced at the time of administration to facilitate its application, and to increase after application so that the composition remains at the site of administration. Also included are mixtures of one or more of these thickening agents.

Permeation enhancing agents are vehicles containing specific agents that affect the delivery of active components through the skin. Permeation enhancing agents are generally divided into two classes: solvents and surface active compounds (amphiphilic molecules). Examples of solvent permeation enhancing agents include alcohols (e.g., ethyl alcohol, isopropyl alcohol), dimethyl formamide, dimethyl sulfoxide, 1-dodecylazocyloheptan-2-one, N-decyl-methylsulfoxide, lactic acid, N,N-diethyl-m-toluamide, N-methylpyrrolidone, nonane, oleic acid, petrolatum, polyethylene glycol, propylene glycol, salicylic acid, urea, terpenes, and trichloroethanol. The surfactant permeation enhancing agent in the present pharmaceutical compositions may be nonionic, amphoteric, cationic, anionic, or zwitterionic. Suitable nonioinic surfactants include poly(oxyethylene)-poly(oxypropylene) block copolymers, commercially known as poloxamers; ethoxylated hydrogenated castor oils; polysorbates, such as Tween 20 or Tween 80. Amphoteric surfactants include quaternized imidazole derivatives, cationic surfactants include cetypyridinium chloride, cationic surfactants include “soap” (fatty acid), alkylsulfonic acid salts (the main component of synthetic detergent, such as linear alkyl benzene sulfonate (LAS)), fatty alcohol sulfate (the main component of shampoo or old neutral detergents), and zwitterionic surfactants include the betaines and sulfobetaines. Also included are mixtures of one or more of these permeation enhancing vehicles.

Injectable formulations can be sterilized, for example, by filtration through a bacterial-retaining filter, irradiation with heat, gamma or e-beam radiation, or by incorporating sterilizing agents in the form of sterile solid compositions which can be dissolved or dispersed in sterile water or other sterile injectable medium prior to use.

### Bioactive Agents

In certain embodiments, the polymers are the only active ingredients in a pharmaceutical composition. In other embodiments, the pharmaceutical composition further comprises one or more bioactive agents. As already mentioned above, a bioactive agent may be associated with the polymer. Alternatively or additionally, a bioactive agent may be added to the composition of the contrast agent and does not form any associations with the contrast agent molecule/macromolecule.

As will be appreciated by one skilled in the art, selection of one or more bioactive agents as component(s) of a pharmaceutical composition will be based on the intended purpose of the pharmaceutical composition (e.g., use in the CT imaging of certain joints or use in other modalities).

In general, the amount of bioactive agent present in a pharmaceutical composition will be the ordinary dosage required to obtain the desired result through local administration. Such dosages are either known or readily determined by the skilled practitioner in the pharmaceutical and/or medical arts.

Examples of bioactive agents that can be present in a pharmaceutical composition of the present invention include, but are not limited to, analgesics, anesthetics, pain-relieving agents, antimicrobial agents, antibacterial agents, antiviral agents, antifungal agents, antibiotics, anti-inflammatory agents, antioxidants, antiseptic agents, antipruritic agents, immunostimulating agents, and dermatological agents. Specific examples of suitable bioactive agents are provided and discussed below.

Pain Relieving Agents. A bioactive agent may be selected for its ability to prevent or alleviate pain, soreness or discomfort, to provide local numbness or anesthesia, and/or to prevent or reduce acute post-operative surgical pain. Thus, suitable pain relieving agents include, but are no limited to, compounds, molecules or drugs which, when applied locally, have a temporary analgesic, anesthetic, numbing, paralyzing, relaxing or calming effect.

Analgesics suitable for use in the present invention include non-steroidal, anti-inflammatory drugs (NSAIDs). NSAIDs have analgesic, antipyretic and anti-inflammatory activity. They act peripherally to provide their analgesic effect by interfering with the synthesis of prostaglandin, through cyclooxygenase (COX) inhibition. There are many different types of NSAIDs, including aspirin and other salicylates. Examples include, but are not limited to, ibuprofen, naproxen, sulindac, diclofenac, piroxicam, ketoprofen, diflunisal, nabumetone, etodolac, oxaprozin, and indomethacin. Aspirin is anti-inflammatory when administered in high doses, otherwise it is just a pain killer like acetaminophen. Acetaminophen has similar analgesic and antipyretic effects to the NSAIDs, but does not provide an anti-inflammatory effect. Several of the more potent NSAIDs have been developed into topical products for local administration to painful areas of the body.

Analgesics suitable for use in the present invention also include opioids. As used herein, the term “opioid” refers to any agonists or antagonists of opioid receptors such as the μ-, κ-, and δ-opioid receptors and different subtypes. Examples of opioids include, but are not limited to, alfentanil, allylprodine, alphaprodine, amiphenazole, anileridine, benzeneacetamine, benzoylhydrazone, benzylmorphine, benzitramide, nor-binaltorphimine, bremazocine, buprenorphine, butorphanol, clonitazene, codeine, cyclazocine, desomorphine, dextromoramide, dezocine, diampromide, dihydrocodeine, dihydrocodeine enol acetate, dihydromorphine, dimenoxadol, dimepheptanol, dimethyl-thiambutene, dioxaphetyl butyrate, dipipanone, diprenorphine, eptazocine, ethoheptazine, ethylketocyclazocine, ethylmethylthiambutene, etonitazene, etorphine, fentanyl, hydrocodone, hydromorphone, hydroxypethidine, isomethadone, ketobemidone, levallorphan, levorphanol, lofentanil, loperamide, meperidine, meptazinol, metazocaine, methadone, metopon, morphine, morphiceptin, myrophine, nalbuphine, nalmefene, nalorphine, naltrindole, naloxone, naltrexone, narceine, nicomorphine, norlevorphanol, normethadone, normorphine, norpipanone, opium, oxycodone, oxymorphone, papaveretum, papaverine, pentazocine, phenadoxone, phenazocine, phenoperidine, piminodine, piperidine, pirtramide, proheptazine, promedol, propiram, propoxyphene, remifentanil, spiradoline, sufentanil, tilidine, trifluadom, and active derivatives, prodrugs, analogs, pharmaceutically acceptable salts, or mixtures thereof.

Examples of peptide opioids include, but are not limited to, [Leu⁵]enkephalin, [Met⁵]enkephalin, DynorphinA, Dynorphin B, α-Neoendorphin, β-Neoendorphin, βₕ-Endorphin, Deltorphin II, Morphiceptin, and active derivatives, analogs, pharmaceutically acceptable salts, or mixtures thereof.

Tricyclic antidepressants can be useful as adjuvant analgesics. They are known to potentiate the analgesic effects of opioids (V. Ventafridda et al., Pain, 1990, 43: 155-162) and to have innate analgesic properties (M. B. Max et al., Neurology, 1987, 37: 589-596; B. M. Max et al., Neurology, 1988, 38: 1427-1432; R. Kishore-Kumar et al., Clin. Pharmacol. Ther., 1990, 47: 305-312). Tricyclic antidepressants include, but are not limited to, amitriptyline, amoxapine, clomipramine, desipramine, doxepin, imipramine, nortriptyline, protriptyline, and trimipramine.

Anesthetics that are suitable for use in the practice of the present invention include sodium-channel blockers. Examples of sodium-channel blockers include, but are not limited to, ambucaine, amolanone, amylcaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dyclonine, ecogonidine, ecogonine, etidocaine, euprocin, fenalcomine, formocaine, hexylcaine, hydroxyteteracaine, isobutyl p-aminobenzoate, leucinocaine, levoxadrol, lidocaine, mepivacaine, meprylcaine, metabutoxycaine, methyl chloride, myrtecaine, naepaine, octacaine, orthocaine, oxethazaine, parenthoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, and active derivatives, prodrugs, analogs, pharmaceutically acceptable salts, or mixtures thereof.

Local anesthetics with different pharmacodynamics and pharmacokinetics may be combined in a pharmaceutical composition in order to improve the effectiveness and tolerance of the composition. For example, a composition may comprise an eutectic mixture of lidocaine and prilocaine, or a mixture of lidocaine and tetracaine. It has been reported (see, for example, U.S. Pat. Nos. 5,922,340 and 6,046,187; both incorporated by reference) that co-administration of a glucocorticosteroid and a local anesthetic may prolong or otherwise enhance the effect of local anesthetics. Examples of glucocorticosteroids include dexamethazone, cortisone, hydrocortisone, prednisone, prednisolone, beclomethasone, betamethasone, flunisolide, fluocinolone, acetonide, fluocinonide, triamcinolone, and the like.

Locally acting vasoconstrictive agents are also known to provide effective enhancement of local anesthesia, especially when administered through controlled release. Examples of vasoconstrictor agents include, but are not limited to, catechol amines (e.g., epinephrine, norepinephrine and dopamine); metaraminol, phenylephrine, sumatriptan and analogs, alpha-1 and alpha-2 adrenergic agonists, such as, for example, clonidine, guanfacine, guanabenz, and dopa (i.e., dihydroxyphenylalanine), methyldopa, ephedrine, amphetamine, methamphetamine, methylphenidate, ethylnorepinephrine ritalin, pemoline, and other sympathomimetic agents.

Anti-Infective Agents. Anti-infective agents for use in pharmaceutical compositions of the present invention are compounds, molecules or drugs which, when administered locally, have an anti-infective activity (i.e., they can decrease the risk of infection; prevent infection; or inhibit, suppress, combat or otherwise treat infection). Anti-infective agents include, but are not limited to, antiseptics, antimicrobial agents, antibiotics, antibacterial agents, antiviral agents, antifungal agents, anti-protozoan agents, and immunostimulating agents.

Antiviral agents suitable for use in the present invention include RNA synthesis inhibitors, protein synthesis inhibitors, immunostimulating agents, and protease inhibitors. Antiviral agents include, for example, acyclovir, amantadine hydrochloride, foscarnet sodium, ganeiclovir sodium, phenol, ribavirin, vidarabine, and zidovudine.

Examples of suitable antifungal agents include lactic acid, sorbic acid, Amphotericin B, Ciclopirox, Clotrimazole, Enilconazole, Econazole, Fluconazole, Griseofulvin, Halogropin, Introconazole, Ketoconazole, Miconazole, Naftifine, Nystatin, Oxiconazole, Sulconazole, Thiabendazole, Terbinafine, Tolnaftate, Undecylenic acid, Mafenide, Silver Sulfadiazine, and Carbol-Fushsin.

Antibiotics and other antimicrobial agents include bacitracin; the cephalosporins (such as cefadroxil, cefazolin, cephalexin, cephalothin, cephapirin, cephradine, cefaclor, cefamandole, cefonicid, ceforanide, cefoxitin, cefuroxime, cefoperazone, cefotaxime, cefotetan, ceftazidime, ceftizoxime, ceftriaxone, and meropenem); cycloserine; fosfomycin, the penicillins (such as amdinocillin, ampicillin, amoxicillin, azlocillin, bacamipicillin, benzathine penicillin G, carbenicillin, cloxacillin, cyclacillin, dicloxacillin, methicillin, mezlocillin, nafcillin, oxacillin, penicillin G, penicillin V, piperacillin, and ticarcillin); ristocetin; vancomycin; colistin; novobiocin; the polymyxins (such as colistin, colistimathate, and polymyxin B); the aminoglycosides (such as amikacin, gentamicin, kanamycin, neomycin, netilmicin, paromomycin, spectinomycin, streptomycin, and tobramycin), the tetracyclines (such as demeclocycline, doxycycline, methacycline, minocycline, and oxytetracycline); carbapenems (such as imipenem); monobactams (such as aztreonam); chloramphenicol; clindamycin; cycloheximide; fucidin; lincomycin; puromycin; rifampicin; other streptomycins; the macrolides (such as erythromycin and oleandomycin); the fluoroquinolones; actinomycin; ethambutol; 5-fluorocytosine; griseofulvin; rifamycins; the sulfonamides (such as sulfacytine, sulfadiazine, sulfisoxazole, sulfamethoxazole, sulfamethizole, and sulfapyridine); and trimethoprim.

Other antibacterial agents include, but are not limited to, bismuth containing compounds (such as bismuth aluminate, bismuth subcitrate, bismuth subgalate, and bismuth subsalicylate); nitrofurans (such as nitrofurazone, nitrofurantoin, and furozolidone); metronidazole; tinidazole; nimorazole; and benzoic acid.

Antiseptic agents include benzalkonium chloride, chlorhexidine, benzoyl peroxide, hydrogen peroxide, hexachlorophene, phenol, resorcinol, and cetylpyridinium chloride.

The risk of infection is directly influenced by a suppressed immune system due to disease or medication. Immunostimulating agents are compounds, molecules or drugs that stimulate the immune system of a patient to respond to the presence of a foreign body, for example, by sending macrophages to the infected site(s). Immunostimulating agents suitable for use in the present invention may be selected from a wide range of therapeutic agents, such as interleukin 1 agonists, interleukin 2 agonists, interferon agonists, RNA synthesis inhibitors, and T cell stimulating agents.

Anti-Inflammatory Agents. Anti-inflammatory agents for use in pharmaceutical compositions of the present invention are compounds, molecules or drugs which, when administered locally, have an anti-inflammatory activity (i.e., they can prevent or reduce the duration and/or severity of inflammation; prevent or reduce injury to cells at the injured/damaged site; prevent or reduce damage or deterioration of surrounding tissue due to inflammation; and/or provide relief from at least one of the manifestations of inflammation such as erythema, swelling, tissue ischemia, itching, fever, scarring, and the like).

Anti-inflammatory agents include NSAIDs and steroidal anti-inflammatory agents. Examples of NSAIDs can be found above. Examples of steroidal anti-inflammatory agents include, but are not limited to, aclomethasone dipropionate, flunisolide, fluticasone, budesonide, triamcinolone, triamcinoline acetonide, beclomethasone diproprionate, betamethasone valerate, betamethasone diproprionate, hydrocortisone, cortisone, dexamethason, mometasone furoate, prednisone, methylprednisolone aceponate, and prednisolone.

Anti-inflammatory agents may, alternatively or additionally, be selected from the wide variety of compounds, molecules, and drugs exhibiting antioxidant activity. Antioxidants are agents that can prevent or reduce oxidative damage to tissue. Examples of antioxidants may include, but are not limited to, vitamin A (retinal), vitamin B (3,4-didehydroretinol), vitamin C (D-ascorbic acid, L-ascorbic acid), α-carotene, β-carotene, γ-carotene, δ-carotene, vitamin E (α-tocopherol), β-tocopherol, γ-tocopherol, δ-tocopherol, tocoquinone, tocotrienol, butylated hydroxy anisole, cysteine, and active derivatives, analogs, precursors, prodrugs, pharmaceutically acceptable salts or mixtures thereof.

### Other Bioactive Agents

In certain embodiments, the bioactive agent is a biomolecule that is naturally present in the body and/or that is naturally secreted at an injured or damaged site (i.e., body area) and plays a role in the natural healing process. As will be apparent to those of ordinary skill in the art, variants, synthetic analogs, derivatives, and active portions of these biomolecules can, alternatively, be used in the compositions as long as they exhibit substantially the same type of property/activity as the native biomolecule. Such variants, synthetic analogs, derivatives or active portions are intended to be within the scope of the term “bioactive agents”.

Bioactive biomolecules may be extracted from mammalian tissues and used in pharmaceutical compositions either crude or after purification. Alternatively, they may be prepared chemically or by conventional genetic engineering techniques, such as via expression of synthetic genes or of genes altered by site-specific mutagenesis.

Examples of suitable bioactive biomolecules include cytokines and growth factors. Cytokines and growth factors are polypeptide molecules that regulate migration, proliferation, differentiation and metabolism of mammalian cells. A diverse range of these biomolecules have been identified as potentially playing an important role in regulating healing. Examples of cytokines include, but are not limited to, interleukins (ILs) (e.g., IL-1, IL-2, IL-4 and IL-8), interferons (IFNs) (e.g., IFN-α, IFN-β, and IFN-γ), and tumor necrosis factors (e.g., TNF-α), or any variants, synthetic analogs, active portions or combinations thereof. Examples of growth factors include, but are not limited to, epidermal growth factors (EGFs), platelet-derived growth factors (PDGFs), heparin binding growth factor (HBGFs), fibroblast growth factors (FGFs), vascular endothelial growth factors (VEGFs), insulin-like growth factors (IGFs), connective tissue activating peptides (CTAPs), transforming growth factors alpha (TGF-α) and beta (TGF-β), nerve growth factor (NGFs), colony stimulating factors (G-CSF and GM-CSF), and the like, or any variants, synthetic analogs, active portions or combinations thereof.

Other examples of suitable bioactive biomolecules include proteoglycans, or portions thereof. Proteoglycans are protein-carbohydrate complexes characterized by their glycosaminoglycan (GAG) component. GAGs are highly charged sulfated and carboxylated polyanionic polysaccharides. Examples of GAGs suitable for use in pharmaceutical compositions of the present invention include, but are not limited to, hyaluronan, chondroitin sulfate, dermatan sulfate, heparan sulfate, and keratan sulfate.

Still other examples of suitable bioactive biomolecules include adhesion molecules. Adhesion molecules constitute a diverse family of extracellular and cell surface glycoproteins involved in cell-cell and cell-extracellular matrix adhesion, recognition, activation, and migration. Adhesion molecules are essential to the structural integrity and homeostatic functioning of most tissues, and are involved in a wide range of biological processes, including embryogenesis, inflammation, thrombogenesis, and tissue repair. Adhesion molecules include matricellular proteins (e.g., thrombospondins and tenascins), and cell surface adhesion molecules (e.g., integrins, selectins, cadherins, and immunoglobulins).

### EXAMPLES

The invention, having been generally described, may be more readily understood by reference to the following examples, which are included merely for purposes of illustration of certain aspects and embodiments of the present invention, and are not intended to limit the invention in any way. All headings are for the convenience of the reader and should not be used to limit the meaning of the text that follows the heading, unless so specified.

### Example 1

An efficient synthetic route to one class of cationic iodinated contrast agent building blocks is described herein. The starting material is 5-amino-2,4,6-triiodoisophthalic acid. This starting material was refluxed in thionyl chloride for 6 hours to produce the diacyl chloride (1), which was purified by a simple extraction between ethyl acetate and 1:1 saturated NaCl/saturated NaHCO₃. The diacyl chloride (1) was then added to an excess of ethylenediamine and stirred for 15 hours at room temperature to yield a triiodinated diamine molecule, 2. The new molecule 2 is 59% iodine by weight and bears two primary amines, which are positively charged at physiological pH 7.

### Example 2

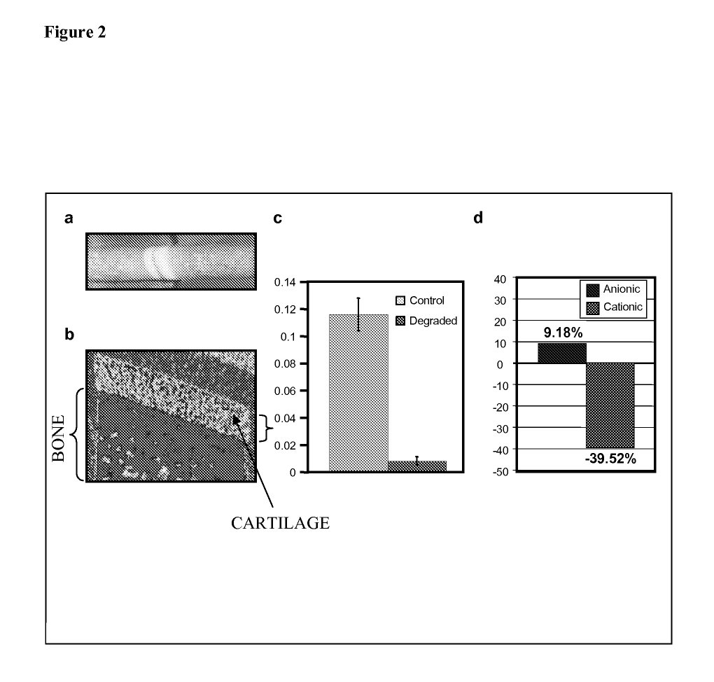

Studies with osteochondral plugs have demonstrated that that cationic contrast agent 2 is capable of enhancing CT images of the cartilage tissue. In one experiment, eight osteochondral plugs (7 mm diameter) were harvested immediately after slaughter from the patella-femoral joint of three mature cows using a water-cooled, cylindrical diamond tipped cutter (See FIG. 2a for an example picture of osteochondral plugs). The plugs were divided into a normal cartilage group (n=4) and a trypsin degraded cartilage group (n=4). The osteochondral plugs of the degraded cartilage group were immersed in trypsin (2 mg/mL in 50 mM Tris, 20 mM CaCl₂, pH=7.8) and incubated for 2 hours at 37° C.

All the plugs were immersed overnight in Cysto Conray II, an anionic, triiodinated contrast agent, diluted in PBS to 16 mg/mL of bound iodine at 4° C. to allow sufficient time for the contrast agent to diffuse into the cartilage. Sequential, 100μ thick, transaxial pQCT images were obtained at 70μ in plane resolution and 300μ inter-slice distance (XCT Research SA+, Stratec) for all the osteochondral plugs. The CT data were imported into Analyze™ (BIR, Mayo Clinic) to create reconstructed 3D images of the osteochondral plugs (see FIG. 2b for a representative image representing one slice of the reconstructed 3D image). A user specified contour was employed to segment the cartilage from the bone. The mean x-ray attenuation coefficient for cartilage was obtained by averaging over all pixels contained in cartilage on the transaxial CT images. The osteochondral plugs were then washed in PBS to remove the anionic contrast agent. The plugs were then re-immersed in the cationic triiodinated contrast agent at the same dilution and conditions used for the anionic contrast agent and repeat CT imaging of the osteochondral plugs was performed.

The GAG content of the articular cartilage was measured using a dimethylmethylene blue (DMMB) assay. A razor blade was used to separate the articular cartilage from the subchondral bone and the cartilage was stored at −20° C. After weighing the hydrated cartilage, it was lyophilized and weighed dry. The cartilage was digested with papain at 65° C. for 24 hours and diluted 10 to 100 times for the assay. Accounting for the dilution, the total GAG weight per mg cartilage dry weight was calculated.

The GAG content measured directly; the x-ray attenuation measured by CT for the anionic and cationic contrast agents were compared for the normal and trypsin degraded articular cartilage plugs using 1-way.ANOVA. Further, the x-ray attenuation measured by CT for both the anionic and cationic contrast agents was expressed as a function of the measured GAG content. The slopes of these relationships were compared to assess the relative sensitivity of these contrast agents to measure differences in the GAG content of articular cartilage.

The GAG content of the normal cartilage measured by the DMMB assay was significantly greater (p<0.05) than that for the trypsin degraded cartilage (FIG. 2c). Similarly, the x-ray attenuation coefficient for the normal osteochondral plugs was greater than that for the trypsin degraded osteochondral plugs (p<0.05) when using the cationic contrast agent. The inverse relation (p<0.05) was observed when using the anionic contrast agent (FIG. 2d). Using the cationic contrast agent, the relative change in x-ray attenuation was directly related and 4.3× more sensitive to the corresponding change in the GAG content of the osteochondral plugs compared to the inverse relationship using the anionic contrast agent.

### Example 3

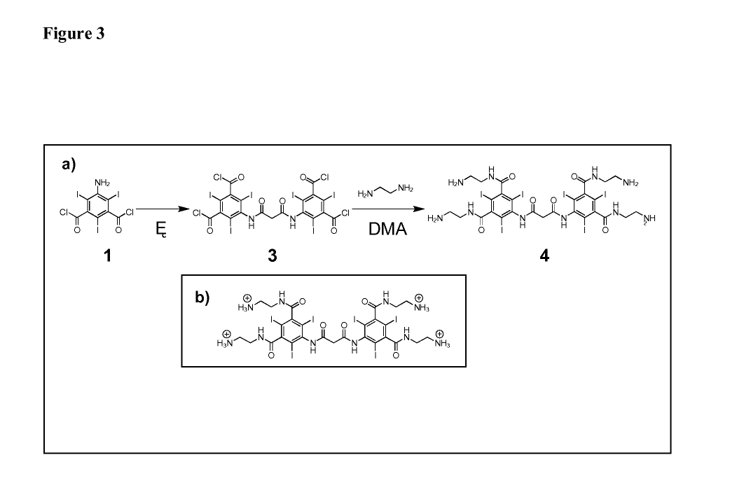

The synthesis of another embodiment of the inventive contrast agents is depicted in FIG. 3. Diacyl chloride 1 (FIG. 3a) was treated with malonyl chloride in refluxing THF to yield the hexaiodo tetrachloride compound 3. The tetraacyl chloride was then treated with ethylene diamine to produce the tetraamine 4. This new molecule is 56% iodine by weight and bears four positively charged primary amines at pH 7.

### Example 4

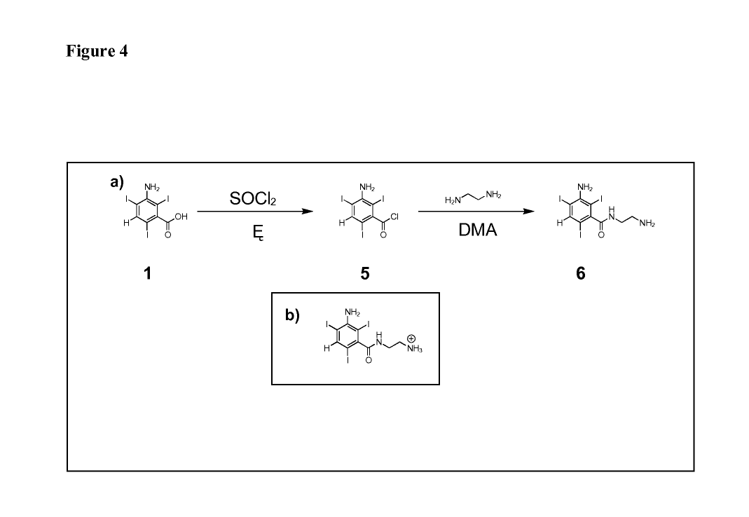

The synthesis of another embodiment of the inventive contrast agents is shown in FIG. 4. 3-amino-2,4,6-triiodobenzoic acid was refluxed in thionyl chloride for 6 hours to produce the acyl chloride, which was purified by a simple extraction between ethyl acetate and 1:1 saturated NaCl/saturated NaHCO₃. The acyl chloride was then added to an excess of ethylenediamine and stirred for 15 hours at room temperature to yield a triiodinated monoamine molecule, 6.

### Example 5

The diffusion characteristics of the contrast agents were evaluated using bovine osteochondral plugs. Two cationic contrast agents, 4 and 6, were compared to a commercially-available singly-charged anionic contrast agent, iothalamate (Cysto Conray II®). Three groups of osteochondral plugs were prepared (n=4)—one for each contrast agent. The plugs were immersed in aqueous contrast agent solution (27 mg of organically-bound iodine/mL, pH 6.8) and removed at 1, 2, 4, 6, 18, and 24 h for imaging using quantitative CT (QCT). Following diffusion into the cartilage, the osteochondral plugs were immersed in saline solution and imaged at the same time points to monitor the diffusion of the contrast agent out of the cartilage.

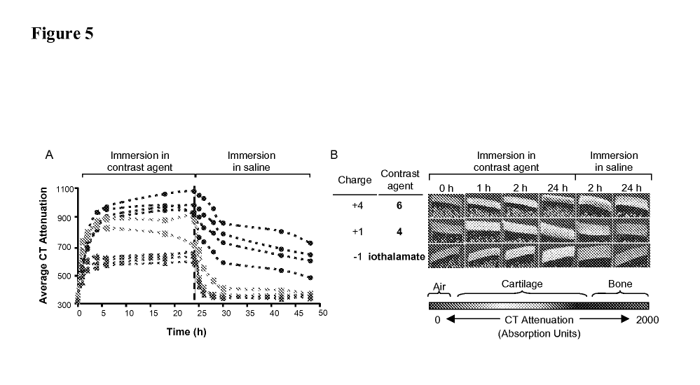

The average CT-based x-ray attenuation was calculated for all three groups of osteochondral plugs, stabilized after ˜6 h (FIG. 5A). When compared to iothalamate, the x-ray attenuations for 4 were ˜30% higher (p=0.03) and those for 6 were ˜60% higher (p=0.001) after equilibration (24 h). After immersion in saline for 6 hours, contrast agent 4 and iothalamate were almost completely removed from the cartilage with the x-ray attenuation of the osteochondral plugs returning to baseline. However, for the plugs immersed in contrast agent 4, the x-ray attenuation of the cartilage decreased by ˜50% over the first 6 hours and by 57% after 24 hours from the saturation values. The negative slope indicates that contrast agent 4 was still diffusing out of the cartilage after 24 h of immersion in saline. The images shown in FIG. 5B reflect the diffusion profile for each contrast agent from the graph in FIG. 5A. The increase and decrease in intensity for each sample can be seen with reference to the colormap scale.

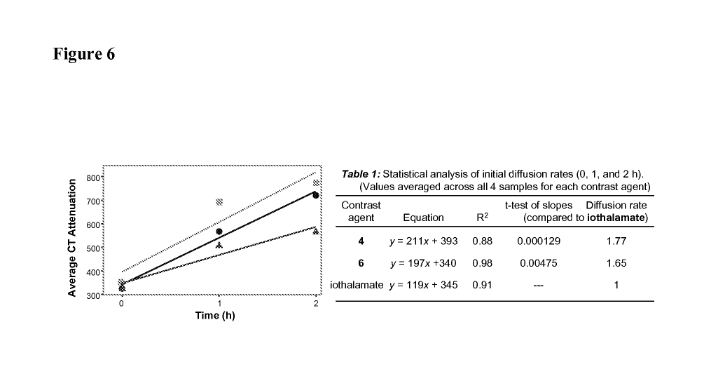

Articular cartilage has a natural anisotropic distribution of GAGs, with low GAG content in the superficial zone and higher GAG content in the middle and deep zones. This anisotropic distribution of GAG was reflected by the variation in x-ray attenuation in the CT images (FIG. 5B). The cationic contrast agents clearly showed higher intensities for deep zone cartilage and lower intensities for superficial zone cartilage. These trends are barely perceptible in the osteochondral plugs imaged with the anionic contrast agent, showing that the anionic contrast agent is less sensitive to subtle changes in the GAG content of the cartilage ECM. In order to determine the initial rates of diffusion into the cartilage tissue, linear regression analyses were applied to the contrast-enhanced CT attenuation data for each osteochondral plug after immersion for 0, 1, and 2 h (Table 1). The slope represents the average rate of diffusion for each contrast agent. Cationic contrast agents 4 and 6 diffuse into cartilage 1.77 and 1.65 times faster, respectively, than the anionic agent iothalamate (FIG. 6).

### Example 6

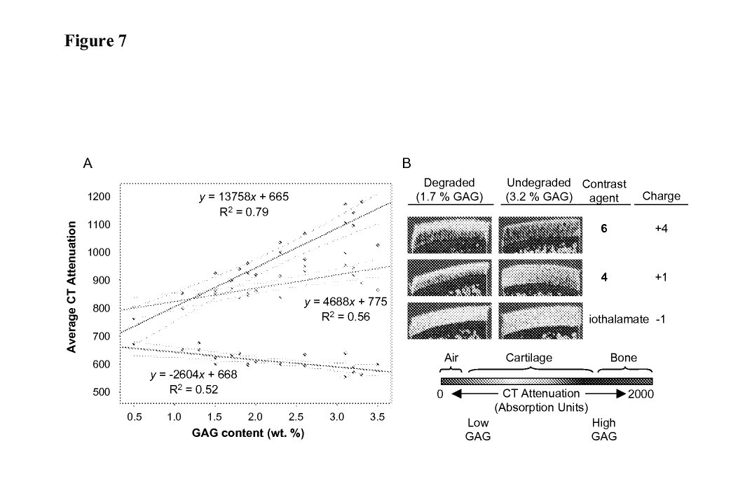

In order to evaluate the ability of the contrast agents to diagnose an OA state, the hyaline cartilage of the osteochondral plugs was enzymatically degraded to mimic the GAG depletion observed in early OA by exposing the hyaline cartilage to different concentrations of the polysaccharide-specific hydrolase, chondroitinase ABC. After degradation, the same set of osteochondral plugs was exposed to each contrast agent consecutively, enabling a direct comparison of the resulting data. The average CT attenuation for each group was plotted as a function of the GAG content obtained by the DMMB assay and subjected to linear regression analysis (FIG. 7A). The CT attenuation of the diffused cationic contrast agents were directly proportional to the GAG content of the hyaline cartilage (contrast 4: R²=0.56, p=0.01; contrast 6: R²=0.79, p<0.0001) whereas the CT attenuation of the diffused anionic contrast agent iothalamate was indirectly related to the GAG content (iothalamate: R²=0.52, p=0.0003). The variation in CT attenuation effected by diffusion of cationic contrast agent 6, with four positive charges, into the hyaline cartilage accounted for 79% of the variation in cartilage GAG, whereas the variation in CT attenuation effected by diffusion of cationic contrast agent 4, with a single positive charge, accounted for 56% of the variation in cartilage GAG. In comparison, the diffusion of anionic contrast agent iothalamate with a single negative charge accounted for 52% of the variation in cartilage GAG. The slope of the regression line represents the sensitivity of contrast enhanced CT attenuation of cartilage to changes in the GAG content of the cartilage. The cationic contrast agents were more sensitive (4: 1.4× and 6: 5.3×) to changes in cartilage GAG content than the anionic contrast agent iothalamate.

FIG. 7B shows representative contrast enhanced CT images for two osteochondral plugs, one where the cartilage is intact, the other where the cartilage has been enzymatically degraded. The color mapped variation in CT attenuation shows increased diffusion of the cationic contrast agents into the intact cartilage with high GAG concentration and less diffusion in the GAG depleted cartilage. The CT attenuation pattern also reflects the diffusion of the cationic contrast agents in proportion to the GAG content of the degraded cartilage. The decreased CT attenuation in the superficial zone cartilage reflects the extent that the chondroitinase was able to penetrate the cartilage and degrade the proteoglycans. By comparison, the change in CT attenuation effected by diffusion of the anionic contrast agent was significantly less obvious.

### Example 7

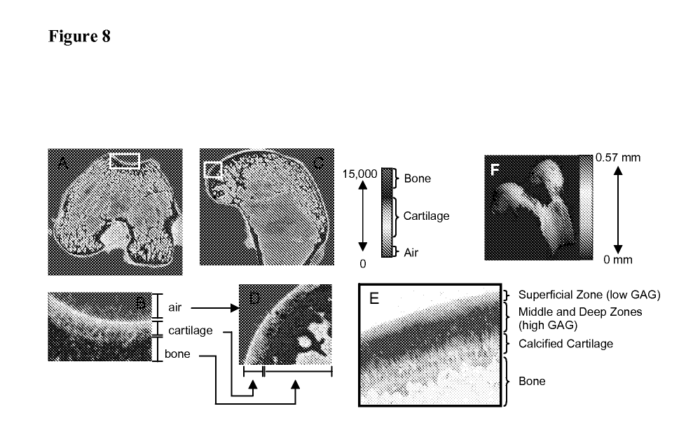

Contrast agent 6 was chosen for use in a rabbit femur study to highlight the ability of the cationic contrast agent to portray the variation in cartilage GAG concentration and cartilage thickness in an intact rabbit femur. An axial slice through the distal femur (FIG. 8A) shows that the cartilage surfaces at the condyles (bottom) and femoral groove (top) can be clearly identified. Additionally, a magnified view of the cartilage surface (FIG. 8B) reveals that the cationic contrast agent is sensitive to the variable distribution of GAG throughout the cartilage surface. The contrast-enhanced CT image was able to define the anisotropic distribution of GAG through the depth of the articular cartilage. A sagittal slice (FIG. 8C) through the distal femur and the corresponding magnified view (FIG. 8D) demonstrates that the CT attenuation increased with increasing depth through the articular cartilage. This result corresponds to the Safranin O stained histological slide (FIG. 8E) that shows the increased GAG content in the deep zone articular cartilage. The contrast enhanced CT image of the femur can also be used to determine the variable thickness of the articular cartilage. FIG. 8F shows a 3D color map of cartilage thickness for the entire distal femur. Cartilage thickness is greater at the femoral groove than at the condyles.

### Example 8

Studies with ex vivo joints and anionic contrast agents have demonstrated that mixing the contrast agent with a viscous solution of hyaluronic acid before injection increases the residence time in the joint and therefore allows for better diffusion of the contrast agent into the cartilage tissue. Six elbow joints from mature New Zealand white rabbits were harvested immediately after sacrifice (Pelfreez Biologicals) and divided into a normal group (n=3) and a trypsin degraded cartilage group (n=3).

Normal Intact Cartilage:
1. Iodinated contrast agent (Cysto-Conray-II) was injected twice at 2-hour intervals.
2. The elbow joint was then lavaged with saline to clear the contrast agent from the joint space.
3. pQCT imaging of the elbow joint was carried out.

Trypsin Degradation of Cartilage:

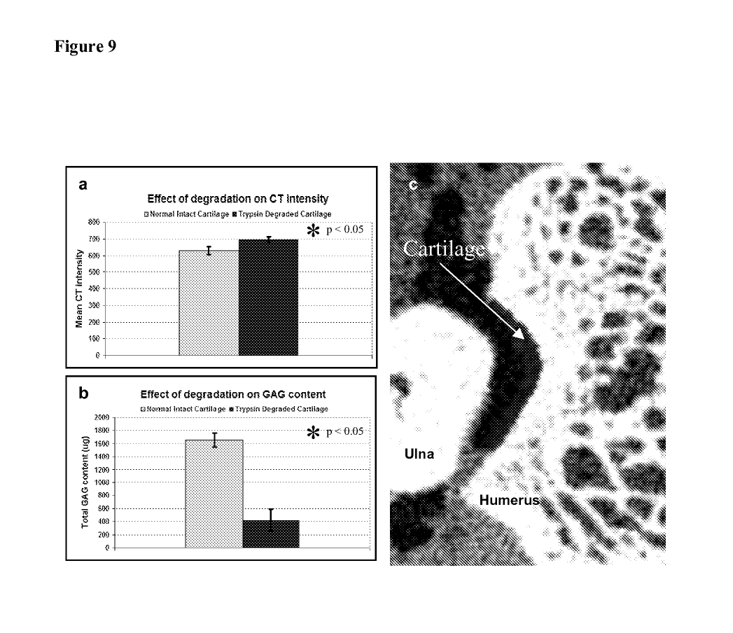

1. One mL of Trypsin (10 mg/mL in 50 mM Tris, 20 mM CaCl₂, and pH 7.8) was injected three times at two hour intervals and the joint was incubated at 37° C. between injections.
2. Iodinated contrast agent (Cysto-Conray-II) was injected twice at 2-hour intervals.
3. The elbow joint was then lavaged with saline to clear the contrast agent from the joint space.
4. pQCT imaging of the elbow joint was carried out.
CT based elbow arthrography: All imaging in this study was performed with Cysto-Conray® II, an anionic triiodinated contrast agent. To obtain the optimum contrast at the bone and cartilage interface, each milliliter of the contrast agent solution was diluted by the addition of 0.3 mL of PBS. The resulting solution was mixed with 5 mg/mL of sodium hyaluronate (Sigma) in PBS to facilitate longer residence times in the joint and aid efficient diffusion through the cartilage tissue. A 22G needle was used to aspirate the synovial fluid from the elbow joint via a direct lateral portal to the elbow joint. 1 mL of contrast agent prepared as above was injected into the joint space under fluoroscopic guidance (OEC mini 6600). Sequential transaxial CT imaging: Images were obtained at a 70 μm in plane resolution and 100 μm slice thickness (inter slice distance=250 microns; number of slices per sample=eight) using a pQCT scanner (Stratec). The image data were imported in Analyze™ (BIR, Mayo Clinic) and a 3D reconstruction of the elbow joint was developed. The cartilage was segmented from the bone using contour-based segmentation (FIG. 9c). A semi-automatic approach was adopted with the user identifying the cartilage bone interface. Biochemical Assay: After imaging, each joint was disarticulated and stored at −20° C. in PBS. Subsequently the cartilage at distal ends of the humeri was digested in papain at 65° C. for 24 hours and analyzed for GAG content using the Dimethyl methylene blue (DMMB) assay. Statistical Analysis: Differences in intensity and GAG content between the control and trypsin treated groups were evaluated using the Student's t-test (α=0.05).

Significant differences in CT intensities and GAG content between the normal and trypsin degraded group were obtained (FIGS. 9b and 9c, p<0.05). The inverse relation between GAG content and CT intensity found here is consistent with studies using anionic iodinated contrast agents. FIG. 9c shows a sample slice with the segmented cartilage shown in red.

### Example 9

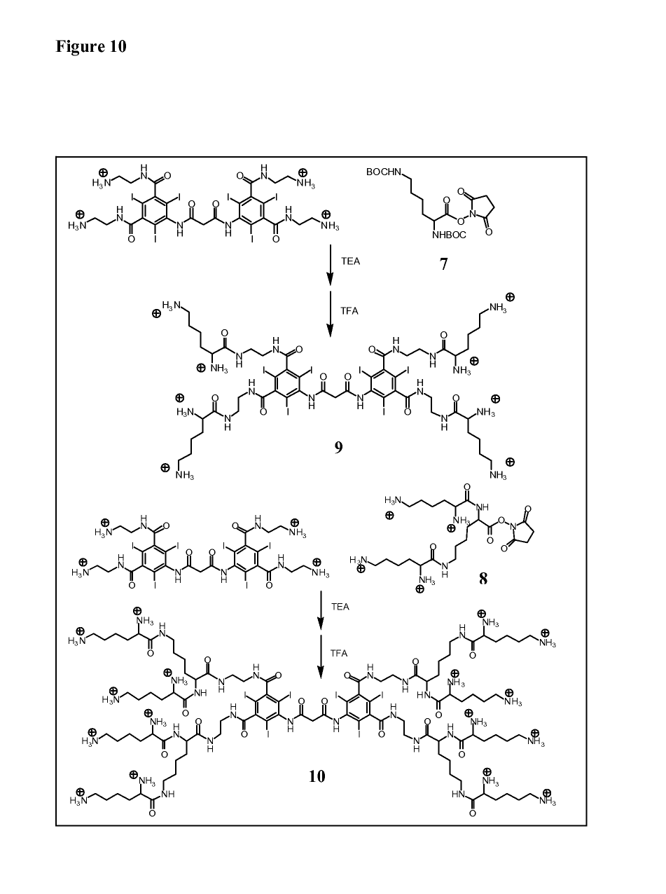

The synthesis of an example of an inventive contrast agent is described in FIG. 10.

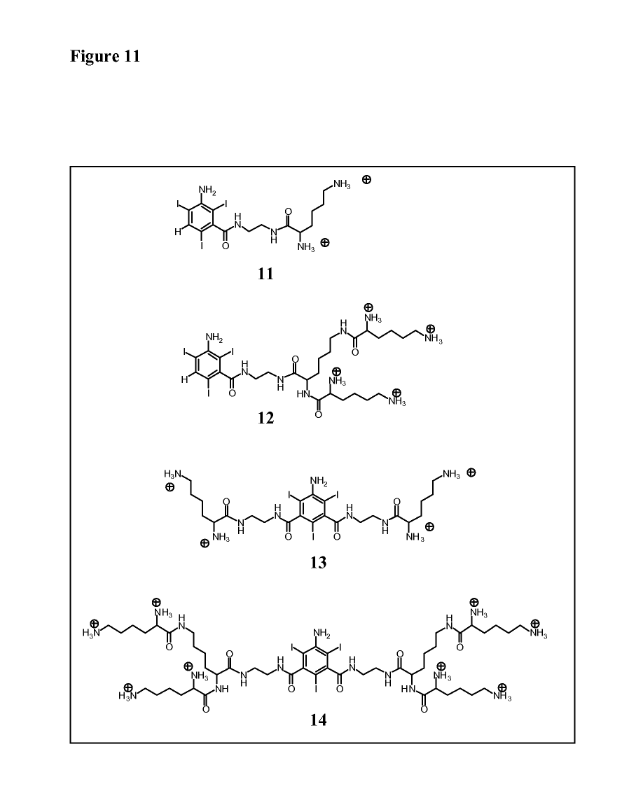

The protonated version of tetraamine 4 is reacted with a Di-Boc protected Lys NHS (7) or a tri-lysine NHS analog (8) in the presence of TEA. Next, the BOC amine groups are cleaved with a dilute solution of TFA. These new molecules (9 or 10) have six iodine atoms per molecule and either bear 8 or 16 positively-charged primary amines at pH 7 as shown in FIG. 10. Using a similar procedure a variety of dendritic multi-cationic contrast agents can be prepared. FIG. 11 shows examples of these molecules.

### Example 10

The hydroxyethyl derivative of contrast agent 4 was synthesized by reacting the acid chloride precursor (3) with a protected amine (16). The amine was prepared by protecting the primary alcohol groups of N,N-bis(2-hydroxyethyl)ethylenediamine (15) with tert-butyldiphenylsilyl chloride. The tetrakis acid chloride (3) was then exposed to 5 equivalents of the protected amino alcohol (16) to yield a TBDPS-protected iodinated contrast agent (17). Deprotection with tetrabutylammonium fluoride afforded the desired molecule (18).

### EQUIVALENTS

Those skilled in the art will recognize, or be able to ascertain using no more than routine experimentation, many equivalents to the specific embodiments of the invention described herein. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations. Such equivalents are intended to be encompassed by the following claims.

All publications and patents mentioned herein, including those references listed below, are hereby incorporated by reference in their entirety as if each individual publication or patent was specifically and individually incorporated by reference. In case of conflict, the present application, including any definitions herein, will control.

1. A contrast agent comprising an iodinated molecule or cationic macromolecule.

2. A contrast agent comprising a structure selected from the group consisting of: 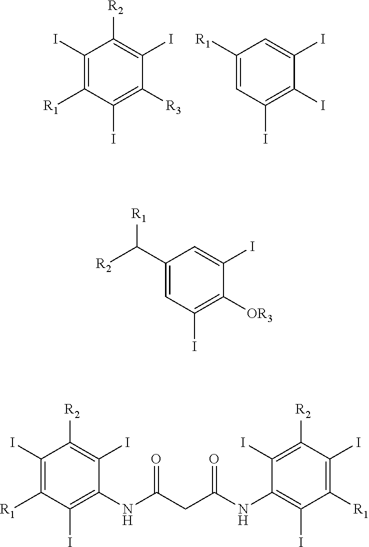
wherein R₁, R₂, and R₃ are either the same or different and selected from the group consisting of H, alkyl, alkenyl, alkynyl, OH, OR′, COOR′, OCOOR′, CONHR′, OCOONHR′, CONHR′₂, NHCONHR′, NHCSNHR′, OCSNHR′, NH₂, NHR′, and NR′₂;
wherein each occurrence of R′ is independently selected from the group consisting of H, an alkyl, an alkenyl, an alkynyl, (CH₂)ₙOH, (CHR″)ₙCH₂R″, (CH₂)ₙNH₂, (CH₂)ₙOR″, (CH₂)ₙNHR″, (CH₂)ₙCOOH, (CH₂)ₙCOOR, CO(CH₂)ₙOR″, COO(CH₂)ₙOR″, COCH(OR″)(CH₂)ₙOR″, COOCH(OR″)(CH₂)ₙOR″, (CHR″)ₙR″, (CH₂CH₂O)ₙR″, ((CH₂)ₘO)ₙR″, an amino acid, a peptide, and a carbohydrate, wherein m and n are integers selected independently from 1 to 2000; wherein each occurrence of R″ is independently selected from the group consisting of H, an alkyl, an alkenyl, an alkynyl, (CH₂)ₙOH, (CH₂)ₙNH₂, OH, OR′″, NH₂, NR″′, SH, SR″′, COOH, COOR″′, CONH₂, (CH₂)ₙNR′″₂, (CH₂)ₙCOOH, (CH₂)ₙCOOR″′, CO(CH₂)ₙOR″, COCH(OR″)(CH₂)ₙOR″, OOCCH₃, amino acid, a peptide, and a carbohydrate, wherein n is an integer from 1 to 2000; and
wherein each occurrence of R″′ is independently selected form the group consisting of H and alkyl.

3. The contrast agent of claim 2, comprising a structure having one or more positive charges.

4-10. (canceled)

11. The contrast agent of claim 2 attached to a polymer.

12. The contrast agent of claim 11, wherein the polymer is a linear polymer or a dendrimer.

13. The contrast agent of claim 11, wherein the contrast agent is covalently attached to the polymer.

14. The contrast agent of claim 11, wherein the contrast agent is non-covalently attached to the polymer.

15. (canceled)

16. (canceled)

17. A composition comprising an effective amount of the contrast agent of claim 2 and an acceptable carrier.

18. The composition of claim 17, further comprising a bioactive agent.

19. The composition of claim 18, wherein the bioactive agent is selected from the group consisting of a growth factor, a cytokine, a small molecule, an analgesic, an anesthetic, an antimicrobial agent, an antibacterial agent, an antiviral agent, an antifungal agent, an antibiotic, an anti-inflammatory agent, an antioxidant, an antiseptic agent, and a combination thereof.

20. A method comprising the step of administering an effective amount of a contrast agent to a mammalian subject in need thereof, wherein the contrast agent comprises a structure selected from the group consisting of: 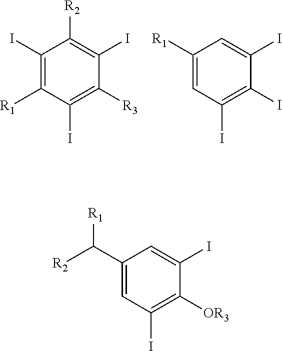
wherein R₁, R₂, and R₃ are either the same or different and selected from the group consisting of H, alkyl, alkenyl, alkynyl, OH, OR′, COOR′, OCOOR′, CONHR′, OCOONHR′, CONHR′₂, NHCONHR′, NHCSNHR′, OCSNHR′, NH₂, NHR′, and NR′₂;
wherein each occurrence of R′ is independently selected from the group consisting of H, an alkyl, an alkenyl, an alkynyl, (CH₂)ₙOH, (CHR″)ₙCH₂R″, (CH₂)ₙNH₂, (CH₂)ₙOR″, (CH₂)ₙNHR″, (CH₂)ₙCOOH, (CH₂)ₙCOOR, CO(CH₂)ₙOR″, COO(CH₂)ₙOR″, COCH(OR″)(CH₂)ₙOR″, COOCH(OR″)(CH₂)ₙOR″, (CHR″)ₙR″, (CH₂CH₂O)ₙR″, ((CH₂)ₘO)ₙR″, an amino acid, a peptide, and a carbohydrate, wherein m and n are integers selected independently from 1 to 2000;
wherein each occurrence of R″ is independently selected from the group consisting of H, an alkyl, an alkenyl, an alkynyl, (CH₂)ₙOH, (CH₂)ₙNH₂, OH, OR′″, NH₂, NR″′, SH, SR″′, COOH, COOR′″, CONH₂, (CH₂)ₙNR′″₂, (CH₂)ₙCOOH, (CH₂)ₙCOOR″′, CO(CH₂)ₙOR″, COCH(OR″)(CH₂)ₙOR″, OOCCH₃, amino acid, a peptide, and a carbohydrate, wherein n is an integer from 1 to 2000; and
wherein each occurrence of R″′ is independently selected form the group consisting of H and alkyl.

21. The method of claim 20, wherein the mammalian subject is a human.

22. The method of claim 20, wherein administering comprises intravenous administration.

23-32. (canceled)

33. A contrast agent comprising a structure selected from the group consisting of: 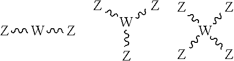
wherein each occurrence of Z is independently selected from the following group consisting of: 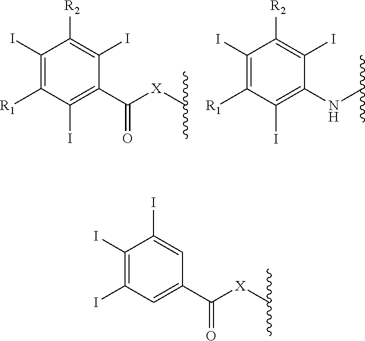
wherein R₁ and R₂ are either the same or different and selected from the group consisting of H, COOR′, CONHR′, CONHR′₂, NH₂, NHR′, NHR′₂;
wherein each occurrence of R′ is independently selected from the group consisting of H, an alkyl, an alkenyl, an alkynyl, (CH₂)ₙOH, (CHR″)ₙCH₂R″, (CH₂)ₙNH₂, (CH₂)ₙOR″, (CH₂)ₙNHR″, (CH₂)ₙCOOH, (CH₂)ₙCOOR, CO(CH₂)ₙOR″, COO(CH₂)ₙOR″, COCH(OR″)(CH₂)ₙOR″, COOCH(OR″)(CH₂)ₙOR″, (CHR″)ₙR″, an amino acid, a peptide, and a carbohydrate, wherein n is an integer from 1 to 2000;
wherein each occurrence of R″ is independently selected from the group consisting of H, an alkyl, an alkenyl, an alkynyl, (CH₂)ₙOH, (CH₂)ₙNH₂, OH, OR′″, NH₂, NR″′, SH, SR″′, COOH, COOR′″, CONH₂, (CH₂)ₙNR′″₂, (CH₂)ₙCOOH, (CH₂)ₙCOOR″′, CO(CH₂)ₙOR″, COCH(OR″)(CH₂)ₙOR″, OOCCH₃, amino acid, a peptide, and a carbohydrate, wherein n is an integer from 1 to 2000;
wherein X is O, S, or NH; and
wherein W is a linking moiety.

34. The contrast agent of claim 33, wherein the linking moiety comprises a covalent linkage bond.

35. The contrast agent of claim 34, wherein the covalent bond is selected from the group consisting of an amide bond, carbamate bond, urea bond, thiourea, Schiff base bond, a peptide ligation, and a carbon-carbon bond.

36. The contrast agent of claim 33, wherein W comprises one of the following structures: 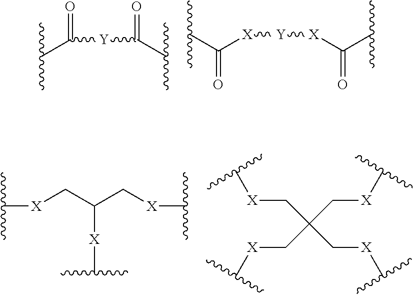
wherein each occurrence of Y is independently selected from the group consisting of alkyl, poly(ethylene glycol), polyethylene, X(CH₂CH₂X)ₙ, and X((CH₂)ₘX)ₙ; wherein each occurrence of X is independently selected from the group consisting of S, O, NH, SH, OH, and NH₂; and
wherein m and n are integers ranging from 1 to 2000.

37. The contrast agent of claim 33, wherein the linking moiety is selected from the group consisting of: COO(CH₂CH₂O)ₙCOO, CONR′(CH₂CH₂O)ₙCONR′, CO(CH₂CH₂O)ₙCO, COO(CH₂CH₂O)ₙCO, CONR′(CH₂CH₂O)ₙCO, (OCH₂CH₂)ₙO, (OCH₂CH₂)ₙNR′, NR′CH₂CH₂(OCH₂CH₂)ₙNR′, CH₂CH₂(OCH₂CH₂)ₙ, and CH₂CH₂(OCH₂CH₂)ₙ, wherein each occurrence of R′ is independently selected from the group consisting of H, an alkyl, an alkenyl, an alkynyl, (CH₂)ₙOH, (CH₂)ₙNH₂, COOH, CONH₂, an amino acid, a peptide, and a carbohydrate, wherein n is an integer from 1 to 2000.

38-44. (canceled)

45. The contrast agent of claim 33 attached to a polymer.

46. The contrast agent of claim 45, wherein the polymer is a linear polymer or a dendrimer.

47. The contrast agent of claim 45, wherein the contrast agent is covalently attached to the polymer.

48. The contrast agent of claim 45, wherein the contrast agent is non-covalently attached to the polymer.

49-65. (canceled)
